(19) United States
(12) Patent Application Publication
(10) Pub. No.: US 2025/0064795 A1
(43) Pub. Date: Feb. 27, 2025

(54) METHODS OF TREATING CORONAVIRUS DISEASE AND COMPOUNDS FOR SAME

(71) Applicants: Board of Supervisors of Louisiana State University and Agricultural and Mechanical College, Baton Rouge, LA (US); SKYMOUNT MEDICAL US INC., New Orleans, LA (US)

(72) Inventors: Adam BESS, Baton Rouge, LA (US); Frej Knut Gosta BERGLIND, Baton Rouge, LA (US); Supratik MUKHOPADHYAY, Baton Rouge, LA (US); Kishor M. WASAN, New Orleans, LA (US); Chris GALLIANO, New Orleans, LA (US)

(21) Appl. No.: 18/724,738

(22) PCT Filed: Dec. 29, 2022
(86) PCT No.: PCT/US2022/082557

§ 371 Date: Jun. 27, 2024

Related U.S. Application Data
(60) Provisional application No. 63/295,142, filed on Dec. 30, 2021.

(51) Int. Cl. A61K 31/4745; A61P 31/14

(52) U.S. Cl. CPC A61K 31/4745; A61P 31/14

(57) ABSTRACT

Topotecan for use in the treatment of COVID-19 and/or its associated symptoms. Also disclosed is a method of treating an individual infected with a coronavirus, wherein said method comprises the steps of: providing Topotecan: and administering said Topotecan to said individual in a dosage amount sufficient to prevent/stabilize/reduce the risks and/or symptoms associated with a coronavirus infection.

### FIELD

This disclosure relates to compounds and compositions for use in the treatment of coronavirus diseases. More particularly, the embodiments of the present disclosure relate to compounds and compositions capable of targeting a mode of interaction between a coronavirus and subject cells.

### BACKGROUND

The appearance of a novel coronavirus, referred to as SARS-CoV-2, on the world stage has affected substantially every population in the world. This virus has afflicted millions of individuals and caused a disease, referred to as COVID-19. COVID-19 can develop into a significant health risk and result in death, which has placed a high strain on healthcare resources and society in general.

SARS-CoV-2 is a single-strand, positive-sense ribonucleic acid (RNA) virus with a similar receptor-binding domain structure to that of SARS-CoV and MERS-CoV. SARS-CoV-2 is transmitted between individuals via airborne droplets accessing nasal mucosa. Within the nasal mucosa SARS-CoV-2 can rapidly reproduce and be shed in nasal secretions (sputum). Sputum can be transmitted to other individuals via airborne droplets, thus repeating the transmission cycle. The SARS-CoV-2 virus can spread between individuals before the onset of symptoms, during the symptomatic period and even after recovery.

The clinical spectrum of the infection is wide, ranging from mild signs of an upper respiratory tract infection to severe pneumonia, multi-organ failure and death. At the onset, SARS-CoV-2 primarily attacks the respiratory system, as it represents the main point of entry into the host, but SARS-CoV-2 also can affect multiple organs of an infected individual. The severity of COVID-19 is typically associated with comorbidities such as, but not limited to hypertension, diabetes, obesity, cardiovascular disease, and/or advanced age that can exacerbate the consequences of COVID-19.

There exists a need for a therapy that is capable of mitigating the impact of COVID-19 in such a manner that it slows down physiological impact on an infected individual.

### SUMMARY

The embodiments of the present disclosure provide one or more therapies for ameliorating and/or inhibiting some or substantially all the risks, symptoms and development of severe disease in a subject infected with a coronavirus. In some embodiments of the present disclosure, the coronavirus is SARS-CoV-2. Given the haste and the magnitude of the SARS-CoV-2 pandemic, it may be advantageous to be identify compounds and compositions for treatment that are already approved for treating other indications.

Some embodiments of the present disclosure relate to a use of Topotecan for ameliorating and/or inhibiting some or substantially all the risks, symptoms and development of severe disease caused by a coronavirus infection.

Some embodiments of the present disclosure relate to a method of treating an individual exposed to or infected with a coronavirus, wherein said method comprises the steps of: providing a therapeutically effective amount of Topotecan; and, administering the therapeutically effective amount of Topotecan to said individual to ameliorate and/or inhibit some or substantially all of the risks, symptoms and development of severe disease in a subject infected with a coronavirus.

Some embodiments of the present disclosure relate to a method of making an Topotecan/target cell complex, the method comprising a step of administering a therapeutically effective amount of Topotecan to a subject, wherein the Topotecan/target cell complex inhibits a coronavirus from: entering into the Topotecan/target cell complex, fusing with the Topotecan/target cell complex and/or replicating within the Topotecan/target cell complex.

Some embodiments of the present disclosure relate to a pharmaceutical composition that comprises Topotecan and at least one excipient.

Without being bound by any particular theory, it is postulated that Topotecan may target the SARS-CoV-2 virus by inhibiting the entry or fusion of the virus with a subject's cell and/or inhibiting replication of the virus once inside a subject's cell. While Topotecan is a known anticancer drug, surprisingly it may also be useful in treating COVID-19.

### BRIEF DESCRIPTION OF THE DRAWINGS

These and other features of the present disclosure will become more apparent in the following detailed description in which reference is made to the appended drawings.

FIG. 1 is a line graph that represents in vitro data from samples, obtained according to embodiments of the present disclosure, regarding efficacy of viral inhibition and toxicity of Topotecan, used according to embodiments of the present disclosure.

FIG. 2 shows a line graph of in vitro data from samples, obtained according to embodiments of the present disclosure, regarding cell viability for various concentrations of Topotecan.

FIG. 3 shows line graph of in vitro data from samples, obtained according to embodiments of the present disclosure, regarding viral inhibition for various concentrations of Topotecan.

FIG. 4A shows a line graph of in vitro data from samples, obtained as a control according to embodiments of the present disclosure, regarding cell viability for various concentrations of Remdesivir.

FIG. 4B shows line graph of in vitro data from samples, obtained as a control according to embodiments of the present disclosure, regarding viral inhibition for various concentrations of Remdesivir.

### DETAILED DESCRIPTION

Unless defined otherwise, all technical and scientific terms used herein have the meanings that would be commonly understood by one of skill in the art in the context of the present description. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

As used herein, the singular forms “a”, “an”, and “the” include plural references unless the context clearly dictates otherwise. For example, reference to “an agent” includes one or more agents and reference to “a subject” or “the subject” includes one or more subjects.

As used herein, the terms “about” or “approximately” refer to within about 25%, preferably within about 20%, preferably within about 15%, preferably within about 10%, preferably within about 5% of a given value or range. It is understood that such a variation is always included in any given value provided herein, whether it is specifically referred to.

As used herein, the term “activity” is used interchangeably with the term “functionality” and both terms refer to the physiologic action of a biomolecule.

As used herein, the terms “agent” and “therapeutic agent” refer to a substance that, when administered to a subject, causes one or more chemical reactions and/or one or more physical reactions and/or or one or more physiological reactions and/or one or more pharmacological reactions and/or one or more immunological reactions in the subject.

As used herein, the term “ameliorate” refers to improve and/or to make better and/or to make more satisfactory.

As used herein, the term “cell” refers to a single cell as well as a plurality of cells or a population of the same cell type or different cell types. Administering an agent to a cell includes in vivo, in vitro, and ex vivo administrations and/or combinations thereof.

As used herein, the term “complex” refers to an association, either direct or indirect, between one or more particles of an agent and one or more target cells or target virions. This association results in a change in the metabolism or functionality of the target cells or target virions. As used herein, the phrase “change in metabolism” refers to an increase or a decrease in the one or more of the targets' production of one or more proteins, and/or any post-translational modifications of one or more proteins. As used herein, the phrase “change in functionality” refers to a difference in physiological function of one or more aspects of the target within an agent/target complex as compared to a target that is not part of such a complex.

As used herein, the terms “dysregulation” and “dysregulated” refer to situations or conditions wherein homeostatic control systems have been disturbed and/or compromised so that one or more metabolic, physiologic and/or biochemical systems within a subject operate partially or entirely without said homeostatic control systems.

As used herein, the term “excipient” refers to any substance, not itself an agent, which may be used in a composition for delivery of one or more agents, and the like to a subject or alternatively combined with . . . one or more carriers and the like (e.g., to create a pharmaceutical composition) to improve its handling or storage properties or to permit or facilitate formation of a dose unit of the composition (e.g., formation of a topical hydrogel which may then be optionally incorporated into a transdermal patch). Excipients include, by way of illustration and not limitation, binders, disintegrants, taste enhancers, solvents, thickening or gelling agents (and any neutralizing agents, if necessary), penetration enhancers, solubilizing agents, wetting agents, antioxidants, lubricants, emollients, substances added to mask or counteract a disagreeable odor, fragrances or taste, substances added to improve appearance or texture of the composition and substances used to form the pharmaceutical compositions. Any such excipients can be used in any dosage forms according to the present disclosure. The foregoing classes of excipients are not meant to be exhaustive but merely illustrative.

As used herein, the terms “inhibit”, “inhibiting”, and “inhibition” refer to a decrease in activity, response, or other biological parameter of a biologic process, disease, disorder or symptom thereof. This can include but is not limited to the complete ablation of the activity, response, condition, or disease. This may also include, for example, a 10% reduction in the activity, response, condition, or disease as compared to the native or control level. Thus, the reduction can be a 10%, 20%, 30%, 40%, 50%, 60%, 70%, 80%, 90%, 100%, or any amount of reduction in between the specifically recited percentages, as compared to native or control levels.

As used herein, the term “medicament” refers to a medicine and/or pharmaceutical composition that comprises an agent and that can promote recovery from a disease, disorder or symptom thereof and/or that can prevent a disease, disorder or symptom thereof and/or that can inhibit the progression of a disease, disorder, or symptom thereof.

As used herein, the term “pharmaceutical composition” means any composition comprising, but not necessarily limited to, one or more agents to be administered a subject in need of therapy or treatment of a disease, disorder, or symptom thereof. Pharmaceutical compositions may include additives such as pharmaceutically acceptable carriers, pharmaceutically accepted salts, excipients and the like. Pharmaceutical compositions may also additionally include one or more further active ingredients such as antimicrobial agents, anti-inflammatory agents, anesthetics, analgesics, and the like.

As used herein, the term “pharmaceutically acceptable carrier” refers to an essentially chemically inert and nontoxic component within a pharmaceutical composition or medicament that does not inhibit the effectiveness and/or safety of the one or more agents. Some examples of pharmaceutically acceptable carriers and their formulations are described in Remington (1995, The Science and Practice of Pharmacy (19th ed.) ed. A. R. Gennaro, Mack Publishing Company, Easton, PA), the disclosure of which is incorporated herein by reference. Typically, an appropriate amount of a pharmaceutically acceptable carrier is used in the formulation to render said formulation isotonic. Examples of suitable pharmaceutically acceptable carriers include, but are not limited to: saline solutions, glycerol solutions, ethanol, N-(1(2,3-dioleyloxy)propyl)-N,N,N-trimethylammonium chloride (DOTMA), dioleolphosphotidylethanolamine (DOPE), and liposomes. Such pharmaceutical compositions contain a therapeutically effective amount of the agent, together with a suitable amount of one or more pharmaceutically acceptable carriers and/or excipients so as to provide a form suitable for proper administration to the subject. The formulation suits the route of administration. For example, oral administration may require enteric coatings to protect the agent from degrading within portions of the subject's gastrointestinal tract. In another example, injectable routes of administration may be administered in a liposomal formulation to facilitate transport throughout a subject's vascular system and to facilitate delivery across cell membranes of targeted intracellular sites.

As used herein, the phrases “prevent”, “prevention of” and “preventing” refer to avoiding the onset or progression of a disease, disorder, or a symptom thereof.

As used herein, the term “subject” refers to any therapeutic target that receives the agent. The subject can be a vertebrate, for example, a mammal including a human. The term “subject” does not denote a particular age or sex. The term “subject” also refers to one or more cells of an organism, an in vitro culture of one or more tissue types, an in vitro culture of one or more cell types, ex vivo preparations, and/or a sample of biological materials such as tissue and/or biological fluids.

As used herein, the term “target cell” refers to one or more cell types within a subject that can interact with a coronavirus by the virus fusing with the outer membrane of the one or more cell types, entering the cell and/or replicating therein. Without being bound to any particular theory, target cells of a subject can include any cells within a subject that express the receptors and/or co-factors required for viral interaction. Examples of these types of cells include but are not limited to: epithelial cells of the upper airways and conducting airways (ciliated and non-ciliated); alveolar epithelial cells (both type 1 and 2); epithelial cells and neurons of the olfactory system; neurons of the central or peripheral nervous system; epithelial cells, enteroctytes and gland cells of the gastrointestinal tract; cells of the blood, including immune effector cells; cardiovascular cells, and renal cells.

As used herein, the term “target virion” refers to one or more viral particles of coronavirus that have the capacity to cause a viral infection within a target cell. In some embodiments of the present disclosure, the viral particles are of one or more variants of SARS-CoV-2.

As used herein, the term “therapeutically effective amount” refers to the amount of the agent used that is of sufficient quantity to ameliorate, prevent, treat and/or inhibit one or more of a disease, disorder, or a symptom thereof. The “therapeutically effective amount” will vary depending on the agent used, the route of administration of the agent and the severity of the disease, disorder, or symptom thereof. The subject's age, weight and genetic make-up may also influence the amount of the agent that will be a therapeutically effective amount.

As used herein, the terms “treat”, “treatment” and “treating” refer to obtaining a desired pharmacologic and/or physiologic effect. The effect may be prophylactic in terms of completely or partially preventing an occurrence of a disease, disorder, or symptom thereof and/or the effect may be therapeutic in providing a partial or complete amelioration or inhibition of a disease, disorder, or symptom thereof. Additionally, the term “treatment” refers to any treatment of a disease, disorder, or symptom thereof in a subject and includes: (a) preventing the disease from occurring in a subject which may be predisposed to the disease but has not yet been diagnosed as having it; (b) inhibiting the disease, i.e., arresting its development; and (c) ameliorating the disease.

As used herein, the terms “unit dosage form” and “unit dose” refer to a physically discrete unit that is suitable as a unitary dose for patients. Each unit contains a predetermined quantity of the agent and optionally, one or more suitable pharmaceutically acceptable carriers, one or more excipients, one or more additional active ingredients, or combinations thereof. The amount of agent within each unit is a therapeutically effective amount.

In embodiments of the present disclosure, the pharmaceutical compositions disclosed herein comprise one or more agents as described above in a total amount by weight of the composition of about 0.1% to about 95%. For example, the amount of the agent by weight of the pharmaceutical composition may be about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%. about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90% or about 95% or more.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also, encompassed within the disclosure, subject to any specifically excluded limit in the stated range. Where the stated range includes one or both of the limits, ranges excluding either or both of those included limits are also included in the disclosure.

The appearance of COVID-19 on the world stage has affected every population in the world, infecting millions of individuals, a number which continues to increase.

Topotecan is conventionally indicated for the treatment of metastatic carcinoma of the ovary and sensitive small cell lung cancer with a mode of action related to binding to a topoisomerase I/DNA complex. Topotecan is a semisynthetic structural analogue of the naturally occurring alkaloid, camptothecin and inhibits topoisomerase 1, an enzyme that functions to facilitate DNA replication during cell division. Topotecan binds covalently to the enzyme as well as to single-strand DNA, eventually causing both single-strand and double-strand DNA breaks that leads to cell death. No data has been published to date, neither clinical trials nor in-vitro studies, to show that Topotecan is an effective treatment for a coronavirus related disease in humans.

Following oral administration, Topotecan is rapidly absorbed with peak plasma concentrations appearing within 1-2 hours and distributes widely to tissues after IV administration. It undergoes pH-dependent hydrolysis to an inactive ring-opened hydroxyl-acid form, which is predominant in plasma. Topotecan is excreted via both bile and urine. The plasma elimination half-life is 2-3 hours.

The dose-limiting toxicity associated with conventional Topotecan therapy is bone marrow suppression. Other commonly reported effects include nausea and/or vomiting, headache, alopecia (hair loss), diarrhea or constipation, abdominal pain, fatigue/weakness, body/back/joint pain, rash, and dyspnea (difficulty breathing). All effects are reversible upon cessation of treatment. The most severe adverse effects reported include interstitial lung disease and severe cases of extravasation and tissue injury.

Based on the mechanism of action and effects noted in repeated-dose and developmental toxicity studies, a potential for Topotecan to adversely affect rapidly dividing cells including sperm and the developing fetus cannot be excluded in the absence of definitive data. In addition, based on mechanism of action and positive genotoxicity findings, Topotecan should be regarded as a probable carcinogen.

Considering the state of the art, potential applications of the use of Topotecan to provide some therapeutic benefits regarding infection with a coronavirus, such as SARS-CoV-2, were identified.

According to the embodiments of the present disclosure, Topotecan may be used in the treatment of COVID-19 and/or symptoms thereof and/or prophylaxis to avoid or mitigate infection following exposure to the SARS-CoV-2 virus.

Some embodiments of the present disclosure relate to a method of treating a subject that has been exposed to the SARS-CoV-2 virus. The steps of the method include providing a therapeutically effective amount of Topotecan and administering that therapeutically effective amount to the subject. The therapeutically effective amount of Topotecan may ameliorate or inhibit some or substantially all of the symptoms of a coronavirus related disease. While the SARS-CoV-2 virus and the related disease of COVID-19 are provided as examples of a coronavirus and a related disease, those skilled in the art will appreciate that the present disclosure is not limited to just SARS-CoV-2 and COVID-19-other coronaviruses and relates diseases are contemplated herein.

Some embodiments of the present disclosure relate to a method of forming a complex between Topotecan and a target cell. The method includes the steps of administering a therapeutically effective amount of Topotecan to the target cell and forming the target cell/Topotecan complex. The complex may be inhibit fusion of the coronavirus with the target cell. The complex may inhibit entry of coronavirus particles into the target cell. The complex may inhibit replication of the coronavirus within the target cell. Some embodiments of the present disclosure relate to administering a therapeutically effective dose of Topotecan to form a target cell/Topotecan complex

Some embodiments of the present disclosure relate to a use of Topotecan for treating a subject who has been exposed to or infected with a coronavirus. Some embodiments of the present disclosure relate to a pharmaceutical composition useful for treating a subject who has been exposed to or infected with a coronavirus. The pharmaceutical composition comprising a therapeutically effective amount of Topotecan and one or more excipients.

In some embodiments of the present disclosure, Topotecan may be used as a therapeutic agent. As a non-limiting example, Topotecan may be provided in one or more medicaments that deliver a dose of between 100 ng and 100 mg of Topotecan.

In some embodiments of the present disclosure, Topotecan may be administered intravenously (IV). When administered by IV, a therapeutically effective amount of Topotecan may be: about 1.5 mg/m² per day for 5 consecutive days which may occur once, twice or three times over first three-week cycle. This dose may fall within a range of about 0.75-2 mg/m² for each administration during the first cycle. A second cycle may occur once, twice, three times of four times over 3 to 4 weeks and it may include about 1.25 mg/m² of Topotecan over 24 hours for 5 consecutive days. A third cycle may be occur once per week over about 4 to 6 weeks and it may include about 1-2 mg/m² per day over 5 consecutive days. The person skilled in the art will recognize that subjects experiencing bone marrow suppression may be treated with reduced or delayed dosing of Topotecan.

In some embodiments of the present disclosure, a target cell may be exposed to a therapeutically effective amount if treated with a concentration of Topotecan of between about 50 nM and about 2200 nM, between about 75 nM and about 2100 nM, between about 100 nM and about 2000 nM, between about 150 nM and about 1800 nM, between about 200 nM and about 1600 nM, between about 300 nM and about 1400 nM, between about 400 nM and about 1200 nM, between about 500 nM and about 1000 nM, between about 600 nM and about 800 nM and all concentration ranges therebetween.

In some embodiments of the present disclosure, the pharmaceutical composition further comprises one or more carriers and/or one or more excipients. In a non-limiting example of the present disclosure, the pharmaceutical composition comprises Topotecan, at least one monoglyceride and/or at least one diglyceride and/or tocopherol polyethylene glycol succinate. In a further non-limiting example of the present disclosure, the pharmaceutical composition may further comprise a lipid-based carrier, such as an emulsion of micro-sized particles and/or nanoparticles. At least one example of suitable nanoparticles are those comprising poly (lactide-co-glycolide).

### EXAMPLES

The DeepDrug™ computational artificial intelligence (AI) system was used to identify Topotecan as likely to be effective against SARS-CoV-2 based on the similarity of Topotecan to antiviral peptides (AVPs) known to target SARS-CoV-1 and other viruses. AVPs are fragments of human proteins that respond to a viral infection by targeting key steps in the viral replication life-cycle including (1) virus binding to the cell surface and internalizing into endosomal compartments (entry), (2) virus being released from endosomal compartments into the cytosol (fusion), and (3) viral protein processing and replication of the viral genome (replication) Protein-Protein binding.

An AI technique was used to generate “fingerprints” for Topotecan and the AVPs in a mathematical representation of all protein interactions in a cell (as described by Grover, A. & Leskovec, J. (2016) node2vec: Scalable Feature Learning for Networks. Kdd, 2016, 855-864). This mathematical representation was created based on the following datasets: AVPdb, a dataset of 2,683 AVPs including 98 from SARS-CoV-1; HPIDB, a dataset of 981 HIV AVPs; hu.map, a dataset of 17.5 million protein-protein interactions; Corum, a dataset of 4,274 mammalian protein complexes; STRING, a dataset of 4,584,628 proteins from 5,090 organisms; DrugBank, a dataset of 13,491 drugs; and BindingDB, a dataset of 846,857 drugs and 7,605 proteins.

An AI technique called a Siamese Network (SNet) was used to compare the fingerprints of Topotecan to the fingerprints of AVPs. SNet predictions were based on a small number of SARS-CoV-1 AVPs that exhibited the strongest antiviral effects. Additionally, SNet provided separate predictions for the three mechanisms of viral infection (e.g., entry, fusion, and replication), which afforded a higher degree of specificity in drug screening. The SNet projected fingerprints into a multidimensional space and calculated distances between them, and the closer the prediction was to zero, the more similar a pair of fingerprints were and the more a drug resembled AVPs. Predictions less than the optimal threshold of 0.63 indicate a significant similarity between the fingerprint of a drug and AVP (i.e., a drug having antiviral effects).

Topotecan received significant (i.e., SNet distances less than 0.63) for all three of the viral mechanisms (e.g., entry, fusion, and replication). Table A below summarizes the SNet prediction data for Topotecan.

TABLE A
SNet Predictions of Topotecan for Entry, Fusion, and Replication.

| Entry Prediction | Fusion Prediction | Replication Prediction |
|---|---|---|
| [Ranking] | [Ranking] | [Ranking] |
| 0.5523 | 0.6017 | 0.5006 |
| [220^{th}] | [246^{th}] | [163^{rd}] |

An assessment of the potential of Topotecan to bind with coronavirus particles was carried out. Using three different mechanism potential binding sites for small molecules, the likelihood of protein-protein binding was determined using an AI system. Using a template of the crystal structure of an essential SARS-CoV-2 protease, the functional centers of the protease inhibitor-binding pocket were identified.

Antiviral peptides known to inhibit the SARS virus were used as targets. By creating a fingerprint of the AVPs, they were then compared to similarly generated fingerprint(s) (embedding) of Topotecan to identify the ones most closely related.

The AVPs used targeted three specific mechanisms: entry, fusion, and replication. The most effective peptides were specifically filtered out and used to create three separate networks based on each peptide's known mechanism of action. This allowed the identification of drugs with certain specificities based on mechanism.

The three mechanisms are relevant for the following reasons. Entry is extremely important because inhibiting viral entry into the cell would reduce the amount of virus that acts on the cell. Likewise, inhibition of replication is important for reducing the amount of viral load generated and spread to other cells after a cell has been infected. Finally, fusion though technically least relevant is worth noting because not all viral entry happens through the standard mechanism. The virus is capable of fusing directly with the membrane of the cell for infection. Though this happens at about 1/10th the rate of the standard entry mechanism, it is still a mechanism which was desirable to use as a focus to attempt to inhibit.

The fingerprints of these specific peptides were created by using the human proteome and a large graph of the proteins involved in all the processes therein. By then comparing these fingerprints to the drug fingerprints, the identification of drugs with a similar (antiviral) effect on the human proteome as the AVPs was carried out.

### First Binding Mechanism

Topotecan was studied to determine its propensity to bind to coronavirus particles according to a first binding mechanism. The interactions where further evaluated by assessing the likelihood that Topotecan would impact the entry of coronavirus into mammalian cells; the fusion of coronavirus particles with mammalian cells; and ultimately the replication of the coronavirus infected cells. Table 1 summarizes the data obtained in this first round of modeling data analysis.

TABLE 1
Results of Protein-Protein modeling data that mimics a first
mechanism of interaction between COVID-19 and Topotecan.
A modelling score greater than 0.25 is a favorable score.

| Corona | Entry | Replication | Fusion |
|---|---|---|---|
| (Rank) | (Rank) | (Rank) | (Rank) |
| 0.2747 | 0.1153 | 0.906 | 0.0128 |
| (117^{th}) | (112^{th}) | (2^{nd}) | (163^{rd}) |

Without being bound to any particular theory, the data collected in the study of the first binding mechanism for Topotecan demonstrated a theoretical propensity to impact the replication of the SARS-CoV-2 virus within a host cell.

### In Vitro Tests

The in vitro efficacy of Topotecan against SARS-CoV-2 (USA-WA1/2020 Isolate) was assessed in human lung cancer (Calu-3) cells. A stock solution of 4 μM of Topotecan was prepared in DMSO and tested at eight concentrations of 5000 nM, 1670 nM, 555.6 nM, 185.2 nM, 61.7 nM, 20.6 nM, 6.9 nM, and 2.3 nM. Calu-3 cells were cultured in 96-well plates and tested in triplicate. A pretreatment/treatment regimen was utilized where cells were incubated with Topotecan for 24±4 hours, cells were then inoculated at a multiplicity of infection (MOI) of 0.005 TCID₅₀ (median tissue culture infectious dose) per cell (200 TCID₅₀/well) with SARS-CoV-2 and incubated for 60-90 minutes. Immediately following incubation, viral inoculum was removed, cells were washed, and wells were overlaid with 0.2 mL Eagle's Modified Essential Media (EMEM) with 2% Fetal Bovine Serum (FBS) containing Topotecan or control articles and incubated in a humidified chamber at 37° C.±2° C. in 5±2% CO2. At 48±6 hours following virus inoculation, cells were fixed and evaluated for the presence of virus by the immunostaining assay (for cytopathic effect (CPE) by the MTT [3-(4,5-dimethylthiazol-2-yl)-2,5-diphenyltetrazolium bromide] assay).

For each well on the assay plate, inhibition of SARS-CoV-2 was calculated as the percentage of reduction of the absorbance value (A₄₅₀ of Topotecan dilution) relative to mean absorbance values from wells designated as positive control (A₄₅₀ virus control; no drug and 0% inhibition) and negative control (A₄₅₀ of cell control; no virus and 100% inhibition) by the following formula:

$$Percent Inhibition = 100 - ( \frac{{A}_{450} of Topotecan dilution - {A}_{450} of cell control}{{A}_{450} of virus control - {A}_{450} of cell control} ) \times 100$$

The effective concentration (IC₅₀) was defined as the concentration of Topotecan that causes 50% reduction of the mean absorbance value of the virus control (0% inhibition) relative to the cell control (100% inhibition). The cytotoxicity of Topotecan at the 8 concentrations was also evaluated as determined by percent inhibition of cell viability.

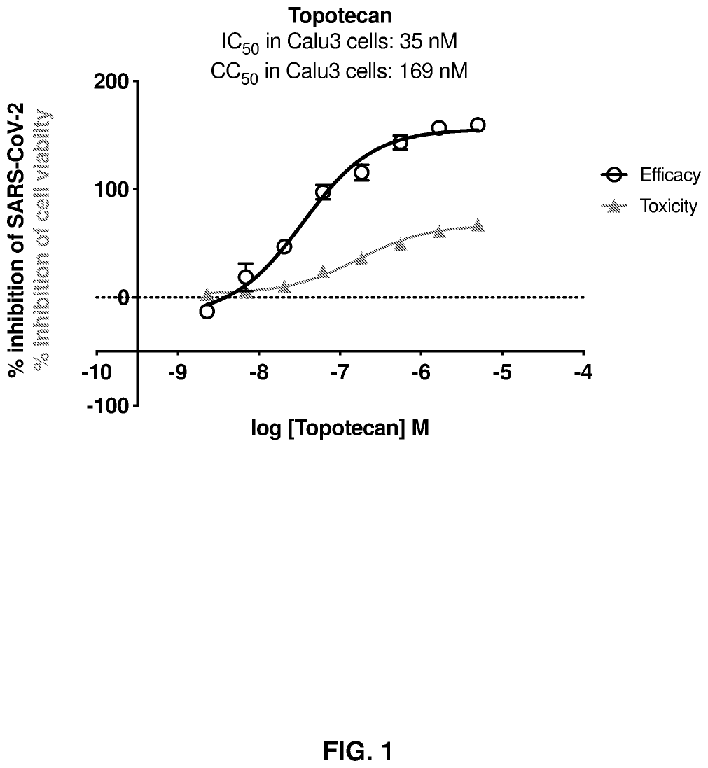

Complete or near complete inhibition of SARS-CoV-2 with an IC₅₀ of 35 nM was determined for Topotecan (see FIG. 1). The cytotoxic concentration (CC₅₀) was 169 nM, indicating a relatively wide margin of safety between the effective concentrations and the cytotoxic concentrations reported in these human lung cells.

### Further Experimental Tests

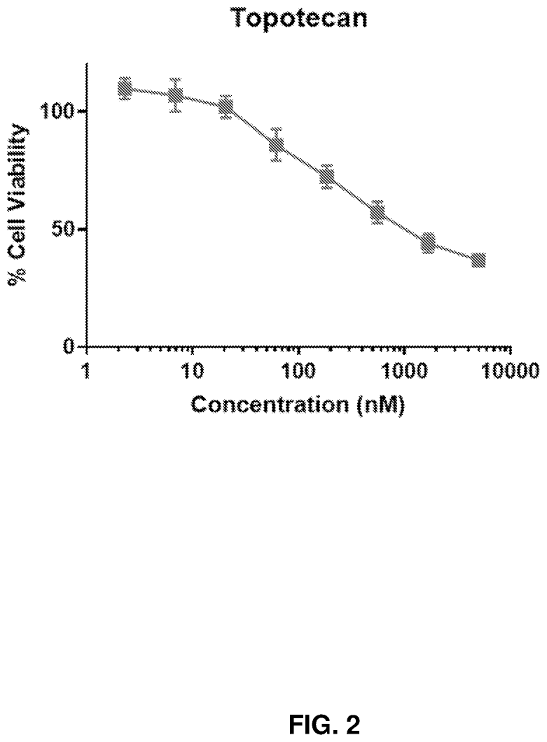

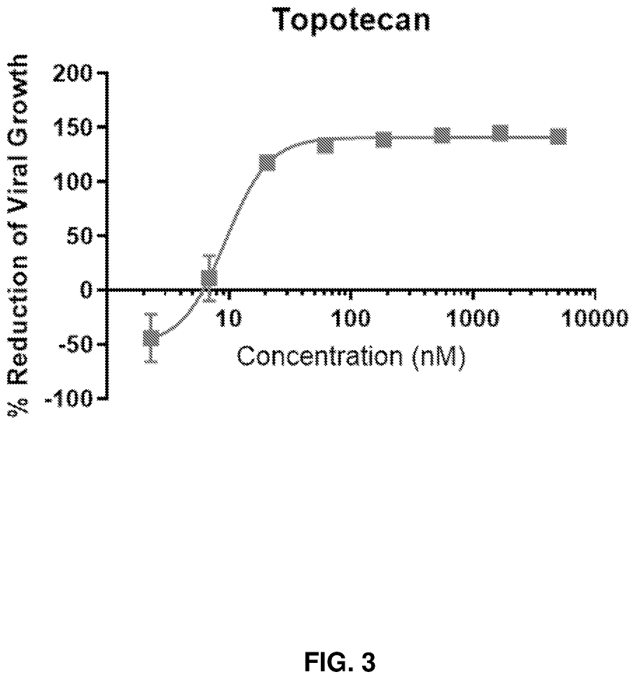

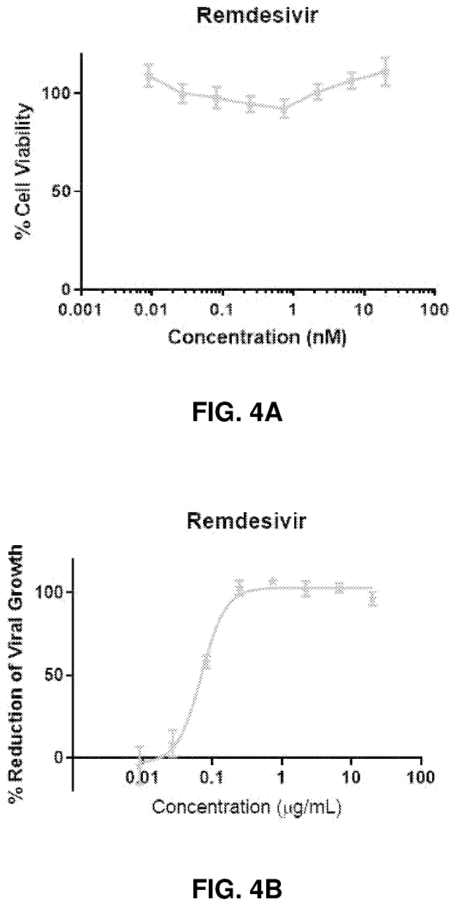

FIG. 2 shows in vitro cell viability data and FIG. 3 shows in vitro viral replicate inhibition data for Topotecan according to the above protocol. FIG. 4A shows in vitro cell viability data and

FIG. 4B shows in vitro viral inhibition data performed according to the above protocol for remdesivir as a control.

Table 2 summarizes IC₅₀ values obtained from the in vitro inhibition data shown in FIG. 3 and FIG. 4B.

TABLE 2
Results of replicate experiments investigating
the IC₅₀ of Topotecan and Remdesivir.

| Name | IC₅₀ (nm) | R² |
|---|---|---|
| Topotecan | 9.362 | 0.9815 |
| Remdesivir | 115.7 | 0.9811 |

Without being bound to any particular theory, the data collected in the study of the IC₅₀ of Topotecan and Remdesivir demonstrated that Topotecan has a significantly lower IC₅₀ compared to Remdesivir.

1. A method of treating a subject exposed to or infected with a coronavirus, the method comprising steps of:
(a) providing a therapeutically effective amount of Topotecan; and
(b) administering the therapeutically effect amount to the subject,
wherein the treating comprises ameliorating and/or inhibiting some or substantially all of the symptoms of a coronavirus-related disease in the subject.

2. The method of claim 1, wherein the coronavirus is SARS-CoV-2 or a variant thereof.

3. A method of forming a target cell/Topotecan complex, the method comprising steps of:
(a) administering a therapeutically effect amount of Topotecan to the target cell; and
(b) forming the target cell/Topotecan complex,
wherein the target cell/complex inhibits fusion of a coronavirus with the target cell, inhibits entry of the coronavirus into the target cell and/or inhibits replication of the coronavirus within the target cell.

4. A method of forming a target virion/Topotecan complex, the method comprising steps of:
(a) administering a therapeutically effect amount of Topotecan to the target virion; and
(b) forming the target virion/Topotecan complex,
wherein the target virion/complex inhibits fusion of target virion with a target cell, inhibits entry of the target virion into the target cell and/or inhibits replication of the target virion within the target cell.

5. The method of claim 3, wherein the therapeutically effective amount is between about 75 nM to about 2100 nM.

6. The method of claim 3, wherein the therapeutically effective amount is between about 100 nM and 2000 nM.

7. The method of claim 3, wherein the coronavirus is SARS-CoV-2 or a variant thereof.

8. The method of claim 4, wherein the target virion is SARS-CoV-2 or a variant thereof.

9. The method of claim 3, wherein the target cell is an epithelial cell of the upper airways and conducting airways (ciliated and non-ciliated); an alveolar epithelial cell (either type 1 or 2); an epithelial cell of the olfactory system, a neuron of the olfactory system; a neuron of the central nervous system, a neuron of the peripheral nervous system; an epithelial cell of the gastrointestinal tract, an enteroctyte of the gastrointestinal tract, a gland cell of the gastrointestinal tract; a white blood cell, a red blood cell, a thrombocyte, an immune effector cell; a cardiovascular cell, and a renal cell.

10. (canceled)

11. (canceled)

12. The method of claim 4, wherein the therapeutically effective amount is between about 75 nM to about 2100 nM.

13. The method of claim 4, wherein the therapeutically effective amount is between about 100 nM and 2000 nM.

14. The method of claim 1, wherein the subject is exposed to the coronavirus.

15. The method of claim 1, wherein the subject is infected with the coronavirus.
